(19) United States
(12) Patent Application Publication
(10) Pub. No.: US 2018/0125520 A1
(43) Pub. Date: May 10, 2018

(54) RETRACTABLE KNIFE

(71) Applicant: SURGICAL SPECIALTIES CORPORATION, Braintree, MA (US)

(72) Inventor: John S. Lehn, JR., Reading, PA (US)

(73) Assignee: SURGICAL SPECIALTIES CORPORATION, Braintree, MA (US)

(21) Appl. No.: 15/579,187

(22) PCT Filed: Jun. 3, 2016
(86) PCT No.: PCT/US2016/035832

§ 371 Date: Dec. 1, 2017

Related U.S. Application Data
(60) Provisional application No. 62/170,652, filed on Jun. 3, 2015.

(51) Int. Cl. A61B 17/3211

(52) U.S. Cl. CPC A61B 17/3211; B26B 1/08; A61B 2017/32113

(57) ABSTRACT

A retractable knife includes a blade that is mounted on a blade mounting element, where the blade mounting element is located within and is slidably engaged with a handle. The blade is moved from a retracted to an exposed position upon pushing a button of the blade mounting element, which results in a cantilever arm bending downwards into an open space, and then pushing the element forward to move the blade outside of the protection afforded by the handle. A slit in the handle guides the movement of the blade mounting element.

### FIELD OF THE INVENTION

The present invention relates generally to knives, and in particular to retractable knives wherein the blade can be moved between an extended position when the knife blade is ready for use, and a retracted position where the knife blade is shielded from contact with the surrounding environment.

This application claims priority from the U.S. provisional application bearing Ser. No. 62/170,652, filed Jun. 3, 2015, the specification of which is hereby incorporated by reference in its entirety.

### BACKGROUND

Reliable and economical guarded knives are needed for a range of surgical and medical procedures and other tasks.

### SUMMARY

In one aspect, the present invention provides a retractable surgical knife. In one embodiment, the knife includes a hollow handle that can be tubular and has both a proximal end and a distal end, as well as a longitudinal axis and a length along the longitudinal axis. The handle has a blade protecting region at the proximal end of the handle. Optionally, the proximal and distal ends of the handle are identical, for example, the handle may be symmetrical about the midpoint of the handle, where the midpoint is an equal distance (equidistant) from the proximal and distal ends of the handle. In addition, the retractable knife has a blade mounting element that is slidably engaged with and substantially, entirely, or partially, located within the tubular handle. By “slidably engaged” it is meant that the blade mounting element can slide forward (toward the proximal end of the knife) and backward (toward the distal end of the knife) within the handle, and that the blade mounting element is engaged with the handle in such a way that the handle provides constraint on the movement of the blade mounting element. The blade mounting element has a longitudinal axis and a length along the longitudinal axis. The blade mounting element can be formed from a single (exactly one) material, such as polypropylene or a material having similar or equivalent flexure properties as polypropylene. The retractable knife includes a blade that is mounted to a proximal end of the blade mounting element. The handle of the knife includes a lengthwise slot connecting a proximal button rest and a distal button rest. Thus, the slot runs in a longitudinal direction of the handle, and at each end or terminus of the slot there is a button rest. The slot is narrower than either of the two button rests. The slot can span a midpoint of the handle as measured along the length of the handle, that is, the slot can span a midpoint that is equidistant from the proximal and distal ends of the handle. The proximal and distal buttons rests can have identical dimensions. The blade mounting element of the knife includes a cantilever arm that is flexible. The blade mounting element can include exactly one (a single) cantilever arm. The top of the cantilever arm, in an unstressed state, has substantially the same height above the longitudinal axis of the blade mounting element as does the surrounding surface of the blade mounting element. In other words, the top surface of the cantilever arm and the top surface of the neighboring portion of the blade mounting element are at the same height. A button sits on top of, i.e., is located on, the cantilever arm, and when the arm is in an unstressed state, open space exists below the cantilever arm. The button is on the free end of the cantilever arm. When the cantilever arm is in a stressed state, for example when someone pushes downward on the button, then a portion of the cantilever arm will move into the formerly open space. The open space abuts a bottom of the cantilever arm and an internal distal side and an internal proximal side of the blade mounting element, and a cutout is located at the internal distal side of the element. The cutout can facilitate flexing of the cantilever arm. The knife can have exactly one (a single) cutout, and not two or more cutouts, that facilitate flexing of the cantilever arm. That is, an underside (bottom) of the cantilever arm is spaced away from and faces an internal distal side and an internal proximal side of the blade mounting element. The cantilever arm has a free end and a connected end. The connected end is connected with a remainder (rest) of the blade mounting element. The cutout is located at the connected end. The cantilever arm can extend from the connected end toward the blade, i.e., in a proximal direction. Alternatively, the cantilever arm can extend from the connected end away from the blade, i.e., in a distal direction. The button includes a neck that rises from the top surface of the cantilever arm, and a thumb rest sits on top of the neck. On either axial side of the neck, there are shoulders that rise above the surface of the cantilever arm. The neck of the button fits through the slot of the handle, to thereby create slideable engagement between the blade mounting element and the handle. The neck and shoulders of the cantilever arm fit snugly in the proximal and distal button rests. The button is slidably engaged with at the slot, the proximal button rest, and/or the distal button rest. Transition of the button from the proximal button rest to the distal button rest concurrently retracts the blade from a fully extended position to a fully protected position within the hollow handle (that can be a tubular handle). That is, when the button is engaged with the proximal button rest, the blade is at a fully extended position. When the button is engaged with the distal button rest, the blade is at a fully protected position within the blade protecting region of the handle.

In an embodiment, the knife only includes (is formed exclusively of) a first half of the hollow handle, a second half of the hollow handle (that can mate to the first half of the hollow handle), the blade mounting element, and the blade. In an embodiment, the blade mounting element does not have two cantilever arms. In an embodiment, the knife does not include a spring. In an embodiment, the knife does not have a retractable blade guard, or the knife has a blade guard that is non-retractable.

The hollow handle can have a substantially cylindrical exterior, or have an exterior substantially of the than of a prism, such as a triangular prism, a square prism, a pentagonal prism, a hexagonal prism, a heptagonal prism, or an octagonal prism. The proximal button rest can be located, so that it is not substantially at the proximal end and not substantially at the distal end of the handle. The distal button rest can be located so that it is not substantially at the proximal end and not substantially at the distal end of the handle. The length of the handle can be greater than the length of the blade mounting element. The longitudinal axis of the handle can be the same as and overlap with the longitudinal axis of the blade mounting element. The handle can include a surface that covers the majority of the handle and a ridge that encircles the lengthwise slot, the proximal button rest, and the distal button rest, and the ridge can extend above the surface of the handle. The button can abut the ridge when the button is located in the proximal button rest or is located in the distal button rest. The handle can have a smooth surface or can have a surface that has a bumpy, wavy, cross-hatched, or scale texture.

In an embodiment, no other parts are used to form the retractable knife, that is, the retractable knife is formed only of the two halves of the handle, the blade mounting element, and the blade.

In another aspect, the present invention provides a method of making a retractable knife. In one embodiment, the method includes providing a blade mounted in a blade mounting element. Optionally, this is provided by providing each of a blade and a blade mounting element and then mounting the blade in the blade mounting element. The method also includes providing two halves of a hollow handle, for example, a right side and a left side (alternatively, a proximal side and a distal side). The blade mounting element is positioned between the two halves of the handle, and the two halves of the handle are brought together and then fixed into place to form the handle. This results in the blade mounting element being held within the handle, for example, in a slidably engaged manner, thereby forming a working retractable knife. In an embodiment, no other parts are used to form the retractable knife, that is, the retractable knife is formed only of the two halves of the handle, the blade mounting element, and the blade.

The details of one or more embodiments are set forth in the description below. Other features, objects, and advantages will be apparent from the description, the drawings, and the claims.

### BRIEF DESCRIPTION OF THE DRAWINGS

Features of the present disclosure, its nature, and various advantages will be apparent from the accompanying drawings and the following detailed description of various embodiments.

FIG. 1A is a perspective view of a retractable knife of the invention with the blade retracted into the blade protecting region of the handle.

FIG. 1B is a perspective view of a retractable knife of the invention with the blade extended from the handle.

FIG. 2 is an exploded perspective view of the knife of FIG. 1B, illustrating that the knife may be formed from four parts: a blade, a blade mounting element, and two halves of a handle.

FIG. 3 is an expanded view of the encircled region of the blade mounting element of FIG. 2.

FIG. 4A is a top perspective view of a knife of the present disclosure, with the blade in an extended position.

FIG. 4B is a side cross-sectional view of the knife configuration shown in FIG. 4A.

FIG. 5A is a top perspective view of a knife of the present disclosure, with the blade in a protected position.

FIG. 5B is a side cross-sectional view of the knife configuration shown in FIG. 5A.

Corresponding reference numerals indicate corresponding parts throughout the drawings.

### DETAILED DESCRIPTION OF THE INVENTION

Embodiments of the invention are discussed in detail below. In describing embodiments, specific terminology is employed for the sake of clarity. However, the invention is not intended to be limited to the specific terminology so selected. A person skilled in the relevant art will recognize that other equivalent parts can be employed and other methods developed without parting from the spirit and scope of the invention. All references cited herein are incorporated by reference as if each had been individually incorporated.

The present invention may be understood more readily by reference to the following detailed description of the preferred embodiments of the invention and the Drawings (Figures) included herein. It is to be understood that the terminology used herein is for the purpose of describing specific embodiments only and is not intended to be limiting. It is further to be understood that unless specifically defined herein, the terminology used herein is to be given its traditional meaning as known in the relevant art. Any headings used within this document are only being utilized to expedite its review by the reader, and should not be construed as limiting the invention or claims in any manner.

As used throughout this document, including the claims, the singular form “a”, “an”, and “the” include plural references unless indicated otherwise. For example, “a button” includes one or more buttons, while “a single button” refers to one and only one button, i.e., not two or three or four, etc. buttons. As another example, “a cantilever arm” refers to one or more cantilever arms, while “a single cantilever arm” refers to one and only one cantilever arm, i.e., not multiple cantilever arms. The term “one cantilever arm” refers to one and only one arm, unless the context suggests differently.

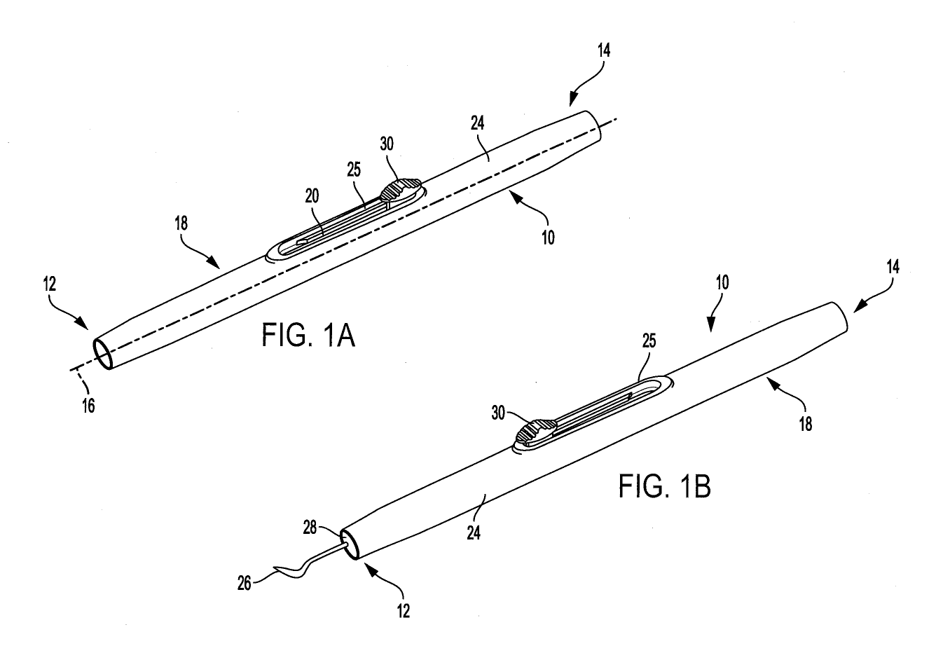

FIG. 1A and FIG. 1B provide two perspective views of an embodiment of a knife 10 of the present invention. FIG. 1A shows the knife 10 in a guarded (protected) configuration, that is, with the blade 26 retracted into the handle 18 of the knife 10. FIG. 1B shows the same knife 10 in an unguarded (unprotected) configuration, that is, with the blade 26 extended.

In the perspective views of FIG. 1A and FIG. 1B, a knife 10 has a proximal end 12 and a distal end 14, as well as a longitudinal axis 16, which for purposes of convenience is shown only in FIG. 1A. The knife 10 includes a hollow handle 18, which may also be referred to as the housing 18, of the knife 10. The hollow portion of the handle 18 may extend along the longitudinal axis 16, so that the handle 18 is open at both the proximal end 12 and the distal end 14, so that the hollow handle 18 has the form of a tube, i.e., is tubular. For example, as depicted in FIG. 1A, the hollow handle 18 may be tubular in form, i.e., be a tubular handle. Alternatively, the distal end 14 of the hollow handle 18 may be closed, so that the hollow handle 18 has the form of a vessel that is open at one end, i.e., the proximal end 12. In FIGS. 1A and 1B the hollow handle 18 depicted has a cylindrical shape, i.e., a circular cross-section. However, in other embodiments the hollow handle 18 may have a non-circular cross-section, e.g., the hollow handle 18 may be in the shape of a triangular tube, square tube, a rectangular tube, a pentagonal tube, a hexagonal tube, a heptagonal tube, an octagonal tube, other polygonal tube, or tube of another convenient cross-section. For example, the exterior of the tube may have the form of a cylinder, triangular prism, square prism, rectangular prism, pentagonal prism, hexagonal prism, heptagonal prism, octagonal prism, or other polygonal prism. The cross-section of the hollow handle 18 can be circular, but is not limited to the form of an ellipse that has zero eccentricity (a circle). For example, the cross-section of the hollow handle 18 can have the form of an ellipse with non-zero eccentricity. The cross section can have an oval form that is nonelliptical or can have another form. The cross section of the hollow handle 18 can be that of a regular polygon, but is not limited to this. For example, the cross section can be that of a polygon that is equilateral, but not equiangular. The cross section can be that of a polygon that is neither equilateral nor equiangular. The cross section can be convex in than, but can also be concave in form. For example, the cross section can have a star shape, for example, the cross section can have the form of a concave octagon, for example, a four-pointed star. The cross section can have a concave shape that is not strictly polygonal, for example, the cross section can have the shape of an oval that has its sides pinched in, for example, to facilitate gripping. The cross section can have a form that is generally polygonal, but has its vertices rounded or beveled. The cross section can have any convex or concave form. The interior of the hollow handle 18 can be cylindrical, for example, as is consistent with the circular opening at the proximal end 12 of the knife 10 as shown in FIGS. 1A and 1B. However, as discussed above for the exterior of the hollow handle 18, the cross section of the interior of the hollow handle 18 can be other than circular, for example, it can be elliptical, oval, polygonal, or have any other convex or concave shape. The cross section of the interior of the hollow handle 18 can have a similar shape as the cross section of the exterior of the hollow handle 18, or it can have a different shape.

Also shown in FIG. 1A and FIG. 1B is that the hollow handle 18 is tapered at each of its proximal end 12 and distal end 14. The taper may be present on the exterior of the hollow handle 18, but not present on the interior of the hollow handle 18 of the knife 10. In one embodiment, the knife 10 includes one tapered end. In another embodiment, the knife 10 includes two tapered ends. In another embodiment, the knife 10 has no tapered ends. The tapering is an example of how the exterior and/or the interior of the hollow handle 18 need not have the strict form of an “extrusion” of a two-dimensional cross section such as a circle, ellipse, or polygon. As another example, the exterior of the hollow handle 18 may have a generally cylindrical form, but near to or at the midpoint along the longitudinal axis be pinched inward, to have an “hourglass” shape, for example, to facilitate holding the knife 10. For example, the interior of the hollow handle 18 may have the form of a truncated cone.

The hollow handle 18 can include a handle slit 20, sometimes referred to as a handle slot 20, which extends in a lengthwise manner along (i.e., along the longitudinal axis 16 of) the handle 18. The slit 20 is an opening in the handle 18, where that opening can be longer than it is wide. In terms of length the slit 20 extends along the longitudinal axis 16 of the handle 18 as defined by the direction from the proximal end 12 to the distal end 14 of the knife 10. The slit 20 has a width, where width is defined as the direction perpendicular to the handle length (i.e., perpendicular to the longitudinal axis 16). Optionally, the slit 20 has a constant width along its entire length. The depth of the slit 20 is substantially the thickness of the wall of handle 18. In an embodiment, the slit 20 occupies a length of from about 0.75 to about 2.25 inches, or from about 1 to about 2 inches, or from about 1.25 to about 1.75, or about 1.5 inches of the length of the handle 18. In an embodiment, the slit 20 does not extend to either of the proximal end 12 or distal end 14 of the knife. In an embodiment, the slit 20 does not extend to substantially the proximal end 12 or to substantially the distal end 14 of the knife.

In an embodiment, the slit 20 is centered at, or at about, the midpoint of the length (along the longitudinal axis 16) of the knife 10. The midpoint of the knife refers to a point that is equidistant from the proximal 12 and distal 14 ends of the handle 18. Positioning the slit 20 over the midpoint of the knife 10 can have advantages. For example, when the knife 10 is in use, the operator's fingers may be located near the proximal end 12 of the knife 10. By placing the slit 20 in the middle of the handle 18, the slit 20 and its accompanying button 30, as discussed below, are away from, and not in the way of, the operator's fingers. In addition, when the slit 20 is symmetrically positioned over the midpoint of the handle 18, then the handle 18 itself may be completely symmetrical, i.e., the distal 12 and proximal 14 ends of the handle 18 are equivalent. This can be advantageous, because, when the knife 10 is assembled, either end of the handle 18 can provide the proximal end 12 of the knife 10. This symmetry makes it easier to assemble the knife 10, because the assembling process does not need to determine which end of the handle 18 should become the proximal end 12 of the knife 10.

The handle 18 has an exterior surface 24 that covers most of the handle, the exception being the slit 20 and optionally, a region 25 that is adjacent to the slit 20 as shown in FIG. 1A and FIG. 1B. The exterior surface 24 may be smooth. In one embodiment, the exterior surface 24 includes tactile features (a texture), for example, small irregular protrusions that impart a roughness to the surface 24 as shown in FIGS. 1A and 1B. Alternatively, the texture may have the form of regularly spaced and formed protrusions. For example, the texture may be a bumpy texture (with regularly or irregularly spaced and/or formed bumps), a wavy texture, a cross-hatched texture, or a scale texture. A scale texture may be formed like the scales of a fish, for example, so that of a scale feature, the portion that is closer to the proximal end 12 is nearer to the longitudinal axis 16, whereas the portion that is farther from the proximal end 12 is farther from the longitudinal axis 16. For example, such a scale texture may inhibit the operator's fingers from slipping toward the proximal end 12 of the knife 10, but allow for the operator's fingers to relatively easily slide toward the distal end 14 of the knife 10. The exterior surface 24 may have any other texture. Although FIG. 1A and FIG. 1B show a region (a ridge) 25 that is raised above the surrounding surface 24, this is an optional feature of the retractable knife 10 of the present disclosure. Optionally, the region that is adjacent to the slit 20 may be identical to the rest of the exterior surface 24 of the handle 18.

The knife 10 also includes a blade 26 which is visible in the extended blade view of FIG. 1B, although it is contained within the handle 18 of the retracted blade view of FIG. 1A and accordingly cannot be seen in FIG. 1A. The blade 26 is attached to a blade mounting element 28. The blade mounting element 28 is slidably engaged with the handle 18, so that when the mounting element 28 is slid forward, i.e., slid toward the proximal end 12 of the knife 10, the blade 26 extends out from the proximal end 12 of the handle 18 and the knife 10 has the configuration shown in FIG. 1B. When the mounting element 28 is slid backward, i.e., toward the distal end 14 of the knife 10, then the blade 26 retracts into the proximal end 12 of the handle 18 and the knife 10 has the configuration shown in FIG. 1A. Manipulation of the button 30 facilitates the sliding of the mounting element 28 in a back and forth direction.

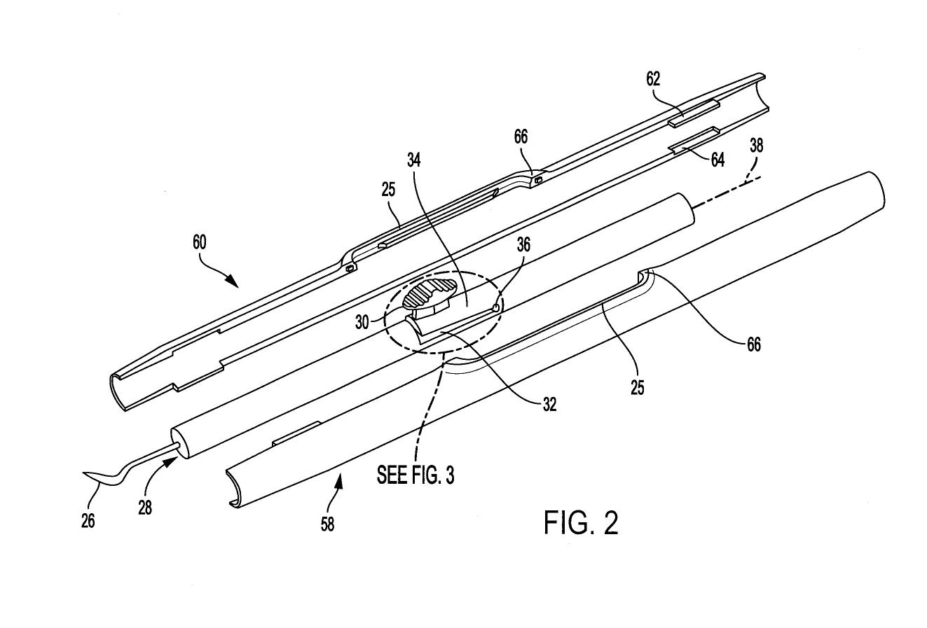

The blade mounting element 28 and the operation thereof to extend and retract the blade 26 when using the knife 10 may be further understood by reference to FIG. 2, which is an exploded perspective view of the extended blade configuration of FIG. 1B. In FIG. 2, the blade 26 is shown attached to the blade mounting element 28. The blade mounting element 28 is sized and shaped to fit within the hollow handle 18, where the handle 18 is shown as having two parts in FIG. 2. For example, when the interior of the handle 18 has a cylindrical shape, then the mounting element 28 may have a substantially cylindrical shape as well, as shown in FIG. 2. The mounting element 28 may have a cross section other than circular that matches the cross section interior of the handle 18 (the shape of the interior of the handle 18 having been discussed above). For example, if the cross section of the interior of the handle 18 has the shape of a pentagon, with the interior of the handle 18 having the form of a pentagonal prism, then the mounting element 28 can have the form of a matching pentagonal prism. Alternatively, if the interior of the handle 18 has the form of an elliptical cone, then the mounting element 28 can have the form of a matching elliptical cone. However, the form of the mounting element 28 need not necessarily match the form of the interior of the handle 18. For example, the interior of the handle 18 may have the form of a hexagonal prism, whereas the form of the mounting element 28 may be that of a triangular prism. When the form of the mounting element 28 does not match the form of the interior of the handle, the form of the mounting element can still be such that the blade mounting element 28 cannot rotate within the handle 18. For example, the vertices of a triangular cross section of the blade mounting element 28 can be located at or near to vertices of a hexagonal cross section of the interior of the hollow handle 18, so that the mounting element 28 cannot rotate within the handle 18.

The mounting element 28 includes a button 30, which is also shown in FIGS. 1A and 1B. In FIG. 2, it can be seen that the button 30 sits above a gap 32 in the mounting element 28. The gap 32 extends from one side, e.g., the left side, to the other side, e.g., the right side, of the mounting element 18, that is, across the entire width of the mounting element 28. The gap 32 extends a distance along the length of the mounting element 28, so as to create a cantilever atm 34. In one embodiment of the knife 10 of the present invention, the blade mounting element 28 has a single cantilever arm 34, that is, it has one and only one cantilever arm 34 (for example, not two cantilever arms 34).

In one embodiment, at the distal end of the gap 32, i.e., at the end of the gap which is closest to the distal end of the knife, the gap 32 may include a cutout 36. This cutout is advantageously present in order to relieve the stress which builds up when the button 30 is depressed and the cantilever arm 34 is forced into the gap 32. The cutout 36 distributes the stress that is created when cantilever arm 34 is pushed into the gap 32. By distributing this stress, the blade mounting element 28 is less prone to breakage, so that the knife 10 will have a longer functional lifetime. The cutout 36 may also facilitate movement of the button 30 in the downward direction, i.e., toward the longitudinal axis 38 of the mounting element 28.

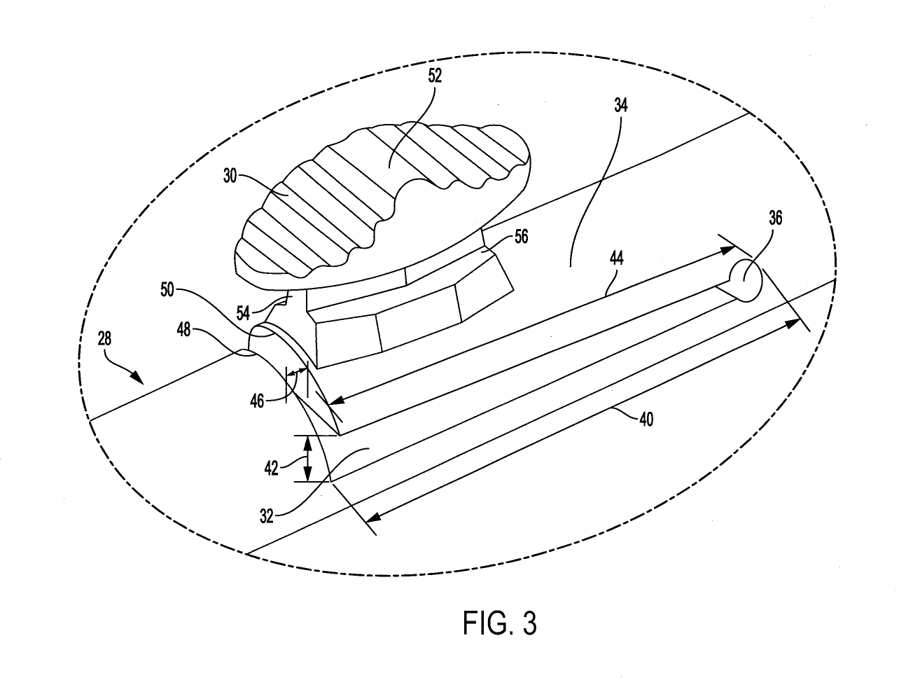

The cantilever arm 34 may be better seen in FIG. 3, which provides an expanded view of the encircled region shown in FIG. 2. In FIG. 3, the button 30 is seen to sit above the gap 32 which defines the underside of a cantilever arm 34. The gap 32 has an optional cutout 36 at the distal end of the gap 32, as discussed previously. The gap runs in a substantially longitudinal direction, running a longitudinal distance 40. The width of the gap 32 is equal to the width of the blade mounting element 28 at the location of the gap, in other words, the gap extends from and includes the sides of the mounting element 28. In one embodiment, the gap 32 runs along the longitudinal axis 38 of the blade mounting element 28, so that when the mounting element 28 has a substantially cylindrical shape, the gap 32 will have a maximum width equal to the maximum width of the mounting element 28. The gap 32 underneath the cantilever arm 34 will have a maximum height 42 when the blade mounting element 28 and the cantilever arm 34 thereof are in an unstressed state. The cantilever arm 34 has a length 44, which may be substantially equal to but cannot exceed the length 40 of the gap 32. In one embodiment, the arm length 44 is less than the gap length 40 by a distance 46, where distance 46 is measured from the proximal end of the cantilever arm 34 and the adjacent side of the blade mounting element 28.

The blade mounting element 28 has a maximum height 48h (not shown) at location 48 above the longitudinal axis 38 of the mounting element 28. The cantilever arm 34, when no forces are being applied to the arm 34, and excluding the button 30, has a maximum height 50h (not shown) at location 50 above the longitudinal axis 38 of the blade mounting element 28. In one embodiment, the distances 48h and 50h are identical. In another embodiment, the distance 50h is longer than the distance 48h, so that at rest, the top of the cantilever arm sits somewhat higher, by a distance 50h-48h, than the top of the adjacent surface of the blade mounting element 28.

In FIGS. 2, 3, 4B, and 5B, the cantilever arm 34 is shown as being connected to the rest (remainder) of the blade mounting element 28 at a connected end, the cantilever arm 34 extending toward the blade 26 and toward the proximal end 12 of the knife 10. The end of the cantilever arm 34 opposite the connected end is a free end (it can move, as described herein). The button 30 is on (in contact with) the free end of the cantilever arm 34. Alternatively, the cantilever arm 34 can be configured, so that it extends from the connected end away from the blade 26 and toward the distal end 14 of the knife 10.

As shown in FIG. 3, the button 30 includes a thumb rest 52 located at the top of the button 30. The operator of the knife 10 will ordinarily engage directly with the thumb rest 52 when changing the knife configuration from a retracted (protected) blade configuration to an exposed (unprotected) blade configuration. Below the thumb rest 52 is a button neck 54, which may also be referred to as a neck 54 or stem 54. In one embodiment, the thumb rest 52 sits directly on top of the neck 54. In one embodiment, the neck 54 sits directly on top of the surface of the cantilever arm 34. The neck 54 will have a maximum width which is not greater than the narrowest width of the handle slit 20 as shown in FIG. 1A and FIG. 1B, in order that the neck 54 may slide through the slit 20. The thumb rest 52 may, and typically will, have a greater maximum width than does the button neck 54.

The button 30 may be located between two shoulders 56 (one shoulder 56 being shown in FIG. 3; the other shoulder 56 being hidden behind the neck 54 in the view of FIG. 3), where the shoulders 56 sit on top of the surface of the cantilever atm 34 and rise up along the sides of the neck 54 towards the thumb rest 52. The shoulders 56 may be sized and shaped to engage with open regions located at the ends of the handle slit 20, which are referred to as button rests, as discussed elsewhere herein.

In one embodiment, the blade mounting element 28 is of unitary construction. In other words, it is made from one continuous piece of material, rather than being formed by joining together two or more material pieces. The blade mounting element 28 may, for example, be formed in a mold, from molten plastic. In another option, the blade mounting element may be formed by a printing technique such as 3D printing or stereolithography.

Returning to FIG. 2, in an embodiment the hollow handle 18 of the knife 10 is formed from two complementary parts, a right-hand part 60 and a left-hand part 58. When the right part 60 and the left part 58 are joined together, they form the handle 18. Optionally, the knife 10 of the present invention is formed from four parts, and optionally is formed from only (exactly) four parts: a knife blade 26, a blade mounting element 28, a right handle part 60, and a left handle part 58. To facilitate the joining together of the two handle parts 58 and 60, the handle parts may include complementary tabs and recesses, where the tab of one housing part will fit into the recess of another housing part. In FIG. 3, one such tab is shown as feature 62, and one such recess is shown as feature 64. In an alternative embodiment, the handle 18 can be formed from a proximal part and a distal part. However, without being bound by theory, an embodiment in which the handle 18 is formed from a right part 60 and a left part 58 may have superior strength and durability with respect to bending stresses resulting from a force imposed on the midpoint of the knife 10 perpendicular to the longitudinal axis 16 that it is opposed to forces imposed on the proximal end 12 and distal end 14 of the knife 10.

In an alternative embodiment, the two handle parts 58 and 60 may be joined by gluing or adhesive bonding or by welding, for example, through the application of heat, ultrasound, vibration, friction, or a solvent.

In one aspect the present invention provides a method of making a retractable knife 10. The method includes providing a blade 26 mounted in a blade mounting element 28. Optionally, this is provided by providing each of a blade 26 and a blade mounting element 28 and then mounting the blade 26 in the blade mounting element 28. The method also includes providing two halves of a handle 18, such as a right part 60 and a left part 58 (alternatively, for example, a proximal part and a distal part). The blade mounting element 28 is positioned between the two halves of the handle 18, and the two halves of the handle 18 are brought together and then fixed into place to form the handle 18. This results in the blade mounting element 28 being held within the handle 18, thereby forming a working retractable knife 10.

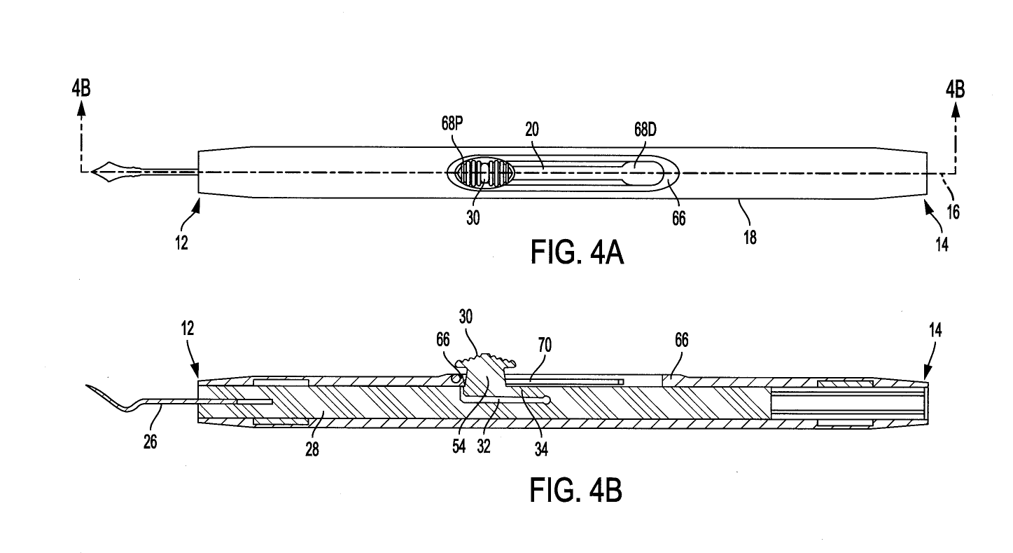

As mentioned previously in connection with FIG. 1A and FIG. 1B, the handle 18 has a region 25 adjacent to the slit 20. That region 25 may be higher than the adjacent surface of the handle, as shown in FIG. 1A and FIG. 1B, and also shown in FIG. 2. Within this region 25, and at one or both of the proximal and distal ends of the slit 20, there may be an enlarged opening 68 (a button rest) (see FIG. 4A, showing an enlarged opening at the proximal end 68P and an enlarged opening at the distal end 68D) where the button 30 will seat when the knife 10 is in the fully retracted or fully extended (exposed) configuration. When the button 30 is seated within an opening 68, the knife 10 will resist movement between a blade-extended and a blade-retracted (contracted) configuration. In order to have the knife 10 change from one configuration to another configuration, the operator (user) will need to push down on the button 30, which will cause the cantilever arm 34 to move into the gap 32, and bring the button neck 54 into alignment with the handle slit 20. Prior to having the button pushed down, the shoulder 56 sits inside and snugly fills the space 68, essentially locking the button into a stationary position. When the cantilever arm 34 is in an unstressed state, the shoulders 56 of the mounting element 28 will abut the sides of a hole (named a button rest 68) that is located at either end of the slit 20.

In one embodiment, when the button 30 is pushed downward, the region 25 is configured such that the underside of the thumb rest 52 will hit against the top of the region 25 (which can be a ridge), and effectively limit the extent to which the cantilever atm 34 may extend into the gap 32. Alternatively, the region 25 can be absent, and the button 30 may be pushed downward until such time as the underside of the cantilever arms abuts against the bottom of the gap 32.

The physical and mechanical properties of the cantilever arm 34 resist movement of the button 30 in the downward direction, and allow the arm 34 to spring back up (return) when pressure is removed from the button 30. The blade mounting element 28 is preferably made from a material which provides the desired resistance to deformation—neither too much nor too little resistance. A suitable material may have a flexural modulus as measured by ASTM D 790 of from about 800 MPa to about 2,400 MPa, or from about 1,200 to about 1,800 MPa, or from about 1,380 to about 1,660 MPa. A suitable material may have a flexural strength as measured by ASTM D 790 of from about 20 MPa to about 80 MPa, or from about 50 to about 65 MPa, or from about 55 to about 58 MPa. A suitable material may have an elongation at break (%) as measured by ASTM D638 of from about 10% to about 25%, or from about 13 to about 20%. A suitable material may have a tensile modulus as measured by ASTM D 638 of from about 1,000 to about 2,000 MPa, or from about 1,400 to 1,800 MPa, or from about 1,590 to about 1,660 MPa. A suitable material may have a tensile strength as measured by ASTM D 638 of from about 25 MPa to about 50 MPa, or from about 35 MPa to about 40 MPa, or about 38 MPa. For example, the blade mounting element 28 may be formed from polypropylene. In an embodiment, the knife 10 does not include an additional spring, such as a coiled, flat spring, or leaf spring, that may be made out of, for example, metal, plastic, or ceramic.

In a preferred embodiment, the ridge of region 25 has a height and location such that it intercepts the downward movement of the thumb rest 52. When the thumb rest 52 is contacted with the top of the ridge in region 25, the shoulders 56 will be lowered such that they are below the inner (underside) surface of the handle 18, and the neck 54 will be at the same height as the slit 20. In this configuration, it can be easy to push the button 30 from one button rest 68 to another button rest 68 as the neck 54 passes through the slit 30, and the shoulders 56 pass beneath the area to either side of the slit 20. When the button 30 has reached a button rest at either end of the slit 20, and pressure is removed from the button 30, the cantilever arm 34 will naturally pop up and position the shoulders 56 within the button rest 68.

In order that the blade 26 does not retract or extend too far, the region 25 may include one or more stops 66, which abut the button 30 when the knife 10 is in the fully extended or fully retracted position. The stop 66 can assist in physically impeding the button 30, and consequently the blade mounting element 28, from moving too far forward (proximally) or backward (distally).

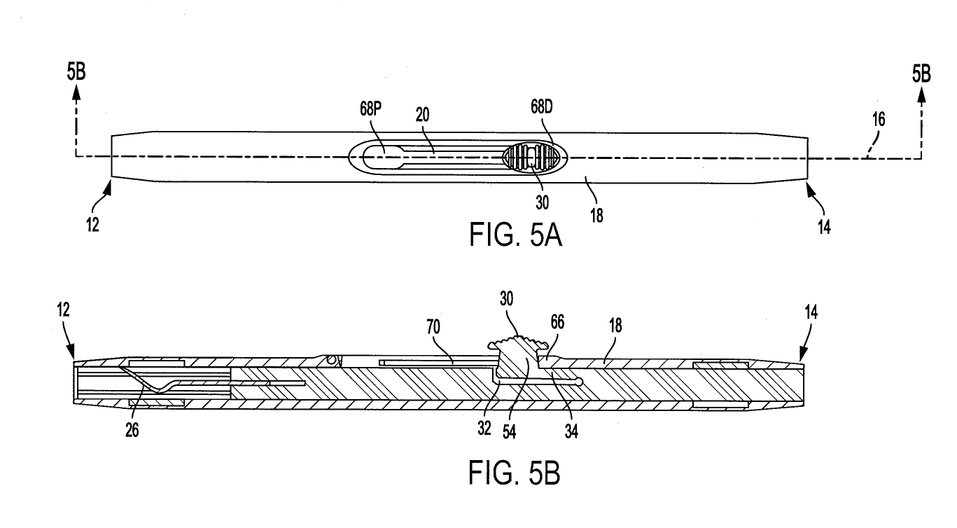

The design and operation of the knife 10 of the present disclosure may be further understood by reference to FIG. 4A and FIG. 4B, where FIG. 4A provides a top view of the knife 10, and FIG. 4B provides a cross-sectional side view of the same knife 10 shown in FIG. 4A. In FIG. 4A and FIG. 4B, the knife 10 is in the extended blade position. The views in these figures may be compared with the corresponding views in FIG. 5A and FIG. 5B which show the knife 10 in the blade-retracted position. In FIGS. 4A, 4B, 5A, and 5B, the knife 10 has a proximal end 12, a distal end 14, a longitudinal axis 16, a button 30, and a slit 20. In FIG. 4A and FIG. 5A it can be seen that the slit 20 terminates, at each of its proximal and distal ends, in a wider opening 68, which is referred to as a button seat 68 (or button rest 68). In the Figures, 68P refers to the button seat (button rest) at the proximal end of the knife 10, while 68D refers to the button seat (button rest) at the distal end of the knife 10.

In operation, with the button 30 located within the button seat 68P as shown in FIGS. 4A and 4B, the blade 26 is in the extended position. The button 30 fits snugly within the button seat 68P due to a variety of factors, each of which may provide some contribution to the snug fit. The primary factor is the size and location of the shoulders 56, which are not readily seen in FIG. 4A, 4B, 5A, or 5B. Another factor is the presence of stop region 66, which abuts the distal side of button neck 54 and precludes the button 30 moving any further in the proximal direction 12. Together, the shoulders 56 and the neck region 54 occupy an area which is substantially equal to the area of a button seat 68 (button rest 68). Another factor is that the housing (handle 18) adjacent to the slit 20 has a thickness (a flange), shown as 70 in FIG. 4B, and that portion of the housing (handle 18) abuts against the distal side of the button neck 54. Another factor is that the cantilever arm 34 is pushing upward against the underside of the housing (handle) 18.

The present invention is illustrated with feature 26 being a blade. The blade 26 is not limited to the design shown in FIG. 1B and FIG. 2 and may be any sharp cutting blade, for example, the blade 26 may be of the type normally seen on a scalpel. In addition, the blade may be substituted with a different item which benefits from being shielded when not in active use, such as a probe, a light source, a needle, an electrode, a sample retriever, or a combination of these and/or other items. In general, the feature 26 may be any feature conveniently located at the end of a handle and needing transitory protection, guarding, or shielding.

The knife 10 may optionally include an identifier, e.g., a logo or trade name, located somewhere on the knife 10.

The present disclosure provides the following exemplary embodiments. A retractable surgical knife having a tubular handle comprising a proximal end and a distal end, the handle comprising a blade protecting region at the proximal end of the handle; a blade mounting element slidably engaged with and substantially located within the tubular handle; and a blade that is mounted to a proximal end of the blade mounting element; wherein the handle comprises a lengthwise slot connecting a proximal button rest and a distal button rest, the proximal and distal buttons rests having identical dimensions, the slot being narrower than either button rest; the blade mounting element comprising a cantilever arm, where a button sits on top of the cantilever arm and open space exists below the cantilever arm, the button being slidably engaged with at least one of the slot, the proximal button rest, and the distal button rest; and transition of the button from the proximal button rest to the distal button rest concurrently retracts the blade from a fully extended position to a fully protected position within the blade protecting region of the tubular handle. Optionally, these are the only functional elements of the knife. In optional features, any one or more of which may be combined, the knife may be further described by: the proximal button rest is not located at substantially the proximal end or at substantially the distal end of the tubular handle; the distal button rest is not located at substantially the proximal end or at substantially the distal end of the tubular handle; the slot spans a midpoint of the handle as measured along a length of the handle; the handle is symmetrical in terms of the proximal and distal ends of the handle being interchangeably able to accept the blade-containing end of the blade mounting element; the blade mounting element has a single cantilever arm; the blade mounting element does not have two cantilever arms; the knife is fainted from only the handle, the blade mounting element, and the blade and does not require any other features to be an operation knife, e.g., the knife does not include a spring and/or the knife does not include a moveable and/or retractable guard; the open space below the cantilever arm abuts each of the bottom of the cantilever arm, an internal distal side of the element, and an internal proximal side of the element, where a cutout is located at the internal distal side of the element to facilitate distributing the stress of the arm moving into and out of the open space; the open space abuts each of the bottom of the cantilever arm, an internal distal side of the element, and an internal proximal side of the element, where a cutout is located at the internal distal side of the element to facilitate the distribution of stress that occurs when the arm is pushed into the open space; the knife has only a single cutout (not two or more cutouts) that facilitate stress distribution; each of the handle and the element has a length, and the length of the handle is greater than the length of the element; each of the handle and the element has a longitudinal axis, and the longitudinal axis of the handle is the same as, i.e., it overlaps with, the longitudinal axis of the element; the handle comprises a surface covering the majority of the handle, and a region or ridge that encircles the lengthwise slot, the proximal button rest, and the distal button rest, where the ridge extends above the surface of the handle; the handle comprises a surface covering the majority of the handle, and a ridge that encircles the lengthwise slot, the proximal button rest, and the distal button rest, where the ridge extends above the surface of the handle, and the button abuts the ridge when the button is located in either of the proximal button rest or the distal button rest.

The embodiments illustrated and discussed in this specification are intended only to teach those skilled in the art the best way known to the inventors to make and use the invention. Nothing in this specification should be considered as limiting the scope of the present invention. All examples presented are representative and non-limiting. The above-described embodiments of the invention may be modified or varied, without departing from the invention, as appreciated by those skilled in the art in light of the above teachings. It is therefore to be understood that, within the scope of the claims and their equivalents, the invention may be practiced otherwise than as specifically described.

The features illustrated or described in connection with one exemplary embodiment may be combined with the features of other embodiments. Thus, any of the various embodiments described herein can be combined to provide further embodiments. Aspects of the embodiments can be modified, if necessary to employ concepts of the various patents, applications and publications as identified herein to provide yet further embodiments.

All of the U.S. patents, U.S. patent application publications, U.S. patent applications, foreign patents, foreign patent applications, and non-patent publications referred to in this specification and/or listed in the Application Data Sheet or Request are incorporated herein by reference, in their entirety. Such documents may be incorporated by reference for the purpose of describing and disclosing, for example, materials and methodologies described in the publications, which might be used in connection with the presently described invention. The publications discussed above and throughout the text are provided solely for their disclosure prior to the filing date of the present application. Nothing herein is to be construed as an admission that the inventors are not entitled to antedate any referenced publication by virtue of prior invention.

In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

What is claimed is:

1. A retractable surgical knife comprising:
a hollow handle comprising a proximal end and a distal end, the hollow handle having a longitudinal axis and a length along the longitudinal axis, the hollow handle comprising a blade protecting region at the proximal end of the handle;
a blade mounting element slidably engaged with and substantially located within the hollow handle, the blade mounting element having a longitudinal axis and a length along the longitudinal axis; and
a blade that is mounted to a proximal end of the blade mounting element;
wherein the hollow handle comprises a lengthwise slot connecting a proximal button rest and a distal button rest, the slot being narrower than the proximal button rest or the distal button rest,
wherein the blade mounting element comprises a cantilever arm,
wherein the cantilever arm has a free end and a connected end, the connected end connected with a remainder of the blade mounting element,
wherein a button is on the free end of the cantilever arm, and the button is slidably engaged with at least one of the slot, the proximal button rest, and the distal button rest.
wherein when the button is engaged with the proximal button rest the blade is at a fully extended position, and when the button is engaged with the distal button rest the blade is at a fully protected position within the blade protecting region of the hollow handle.

2. The knife of claim 1, wherein the hollow handle is tubular.

3. The knife of claim 1, wherein the hollow handle comprises a left half and a right half.

4. The knife of claim 1, wherein the proximal and distal button rests have identical dimensions.

5. The knife of claim 1, wherein the cantilever arm extends from the connected end toward the blade.

6. The knife of claim 1, wherein the hollow handle has a substantially cylindrical exterior.

7. The knife of claim 1, wherein the hollow handle has an exterior substantially of the form of a prism.

8. The knife of claim 1, wherein the hollow handle has an exterior substantially of the form of a prism selected from the group consisting of a triangular prism, a square prism, a pentagonal prism, a hexagonal prism, a heptagonal prism, and an octagonal prism.

9. The knife of claim 1 wherein the proximal button rest is not located at substantially the proximal end or at substantially the distal end of the hollow handle.

10. The knife of claim 1, wherein the distal button rest is not located at substantially the proximal end or at substantially the distal end of the hollow handle.

11. The knife of claim 1 wherein the slot spans a midpoint of the hollow handle as measured along the length of the hollow handle.

12. The knife of claim 1 wherein the blade mounting element comprises a single cantilever arm.

13. The knife of claim 1 wherein the blade mounting element does not have two cantilever arms.

14. The knife of claim 1, which does not comprise a spring.

15. The knife of claim 1,
wherein an underside of the cantilever thin is spaced away from and faces an internal distal side and an internal proximal side of the blade mounting element and
wherein a cutout is located at the internal distal side of the blade mounting element to facilitate flexing of the cantilever arm.

16. The knife of claim 15, wherein the knife has exactly one cutout located at the internal distal side of the blade mounting element.

17. The knife of claim 1, which does not have a retractable blade guard.

18. The knife of claim 1, which has a blade guard that is non-retractable.

19. The knife of claim 1 wherein the length of the hollow handle is greater than the length of the blade mounting element.

20. The knife of claim 1 wherein the longitudinal axis of the hollow handle is the same as and overlaps with the longitudinal axis of the blade mounting element.

21. The knife of claim 1,
wherein the hollow handle comprises a surface covering the majority of the hollow handle and a ridge that encircles the lengthwise slot, the proximal button rest, and the distal button rest and
wherein the ridge extends above the surface of the hollow handle.

22. The knife of claim 21, wherein the button abuts the ridge when the button is located in either of the proximal button rest or the distal button rest.

23. The knife of claim 1, wherein the hollow handle comprises a surface that is smooth.

24. The knife of claim 1,
wherein the hollow handle comprises a surface having a texture selected from the group consisting of a bumpy texture, a wavy texture, a cross-hatched texture, and a scale texture.

25. The knife of claim 1 wherein the handle is symmetrical about a midpoint of the hollow handle, the midpoint being located equidistant from the proximal and distal ends of the hollow handle.

26. The knife of claim 1 wherein the blade mounting element is formed from a single material.

27. The knife of claim 1 wherein the blade mounting element is formed from a single material selected from polypropylene and a material having equivalent flexural properties to polypropylene.

28. A method of making a retractable knife comprising:
providing a blade mounted in a blade mounting element;
providing two halves of a hollow handle;
positioning the blade mounting element between the two halves of the hollow handle;
bringing together the two halves of the hollow handle; and
fixing the two halves of the hollow handle together to form the hollow handle thereby forming the retractable knife.

29. The method of claim 28, wherein the handle is symmetrical about a midpoint of the handle, the midpoint being located equidistant from the proximal and distal ends of the handle.

30. The method of claim 28, wherein the retractable knife is formed only from the two halves of the hollow handle, the blade mounting element, and the blade.
